(19) United States
(12) Patent Application Publication
(10) Pub. No.: US 2017/0215937 A1
(43) Pub. Date: Aug. 3, 2017

(54) THERAPEUTIC TREATMENT DEVICE

(71) Applicant: OLYMPUS CORPORATION, Tokyo (JP)

(72) Inventor: Koichi KUDO, Hachioji-shi (JP)

(73) Assignee: OLYMPUS CORPORATION, Tokyo (JP)

(21) Appl. No.: 15/490,375

(22) Filed: Apr. 18, 2017

Related U.S. Application Data
(63) Continuation of application No. PCT/JP2014/084394, filed on Dec. 25, 2014.

(51) Int. Cl. A61B 18/08

(52) U.S. Cl. CPC A61B 18/085; A61B 2018/00595

(57) ABSTRACT

A therapeutic treatment device heats a living tissue for treatment. The therapeutic treatment device includes a heat conduction plate, an electric resistance pattern, an adhesive sheet and an insulating thin film. The heat conduction plate includes a first principal surface that is brought into contact with the living tissue and a second principal surface. The electric resistance pattern is formed on an insulation substrate and generates heat upon receipt of electric power. The adhesive sheet has a high heat resistance, a high thermal conductivity, and electric insulation properties. The sheet adheres the electric resistance pattern with the heat conduction plate, and transfers the heat generated from the electric resistance pattern to the heat conduction plate. The insulating thin film coveres at least an exposed portion of the electric resistance pattern.

### CROSS-REFERENCE TO RELATED APPLICATIONS

This application is a Continuation Application of PCT Application No. PCT/JP2014/084394, filed Dec. 25, 2014 and based upon prior Japanese Patent Application No. 2013-238338, filed Nov. 18, 2013, the entire contents of all of which are incorporated herein by reference.

### BACKGROUND OF THE INVENTION

1.Field of the Invention

The present invention relates to a therapeutic treatment device.

2.Description of the Related Art

A therapeutic treatment device which treats a living tissue using thermal energy is generally known in the art. The device is configured to transfer heat energy generated by a heater element to a living tissue via a flat heat conduction plate. The heater element, as disclosed in Jpn. Pat. Appln. KOKAI Publication No. 2013-34568, is formed by providing an electric resistance pattern on an insulation substrate. A back surface of the insulation substrate, on which the electric resistance pattern is not formed, is attached to the heat conduction plate.

### BRIEF SUMMARY OF THE INVENTION

According to an aspect of the invention, a therapeutic treatment device heats a living tissue for treatment, the therapeutic treatment device comprising a heat conduction plate including a first principal surface that is brought into contact with the living tissue and a second principal surface opposite to the first principal surface; an electric resistance pattern that is formed on an insulation substrate and generates heat upon receipt of electric power; an adhesive sheet that has a high heat resistance, a high thermal conductivity, and electric insulation properties, adheres the electric resistance pattern with the second principal surface of the heat conduction plate, and transfers the heat generated from the electric resistance pattern to the heat conduction plate; and an insulating thin film covering at least an exposed portion of the electric resistance pattern that is not in contact with the adhesive sheet and the insulation substrate.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic view of a therapeutic treatment device according to each embodiment.

FIG. 2 is a schematic view showing cross sections of an example of configurations of a shaft and a holding section of an energy treatment tool of the embodiments; (A) shows a state in which the holding section is closed, and (B) shows a state in which the holding section is opened.

FIG. 3 is an overall perspective view of a first holding member according to a first embodiment of the present invention.

FIG. 4 is an overall perspective view showing an assembly structure of the first holding member of the therapeutic treatment device.

FIG. 5 is a cross-sectional view of the first holding member.

FIG. 6 is a view for explaining a ratio of lengths in a transverse direction of members that constitute a first holding member of a second embodiment.

FIG. 7 is an overall perspective view of a sub-assembly (1) constituting a first holding member according to a third embodiment of the present invention.

FIG. 8 is a cross-sectional view showing a state in which an insulator thin film is formed on the sub-assembly (1) constituting the first holding member according to the third embodiment of the present invention.

FIG. 9 is a cross-sectional view of the first holding member.

FIG. 10 is a flowchart showing procedural steps of assembling of a therapeutic treatment device according to the third embodiment.

FIG. 11 is an overall perspective view of an electrothermal conversion element constituting a first holding member according to a fourth embodiment of the present invention.

FIG. 12 is a cross-sectional view showing a state in which an insulator is formed entirely on the electrothermal conversion element.

FIG. 13 is a cross-sectional view of the first holding member.

FIG. 14 is a flowchart showing procedural steps of assembling of a therapeutic treatment device according to the fourth embodiment.

### DETAILED DESCRIPTION OF THE INVENTION

A therapeutic treatment device according to first to fourth embodiments of the present invention will be described below with reference to the drawings.

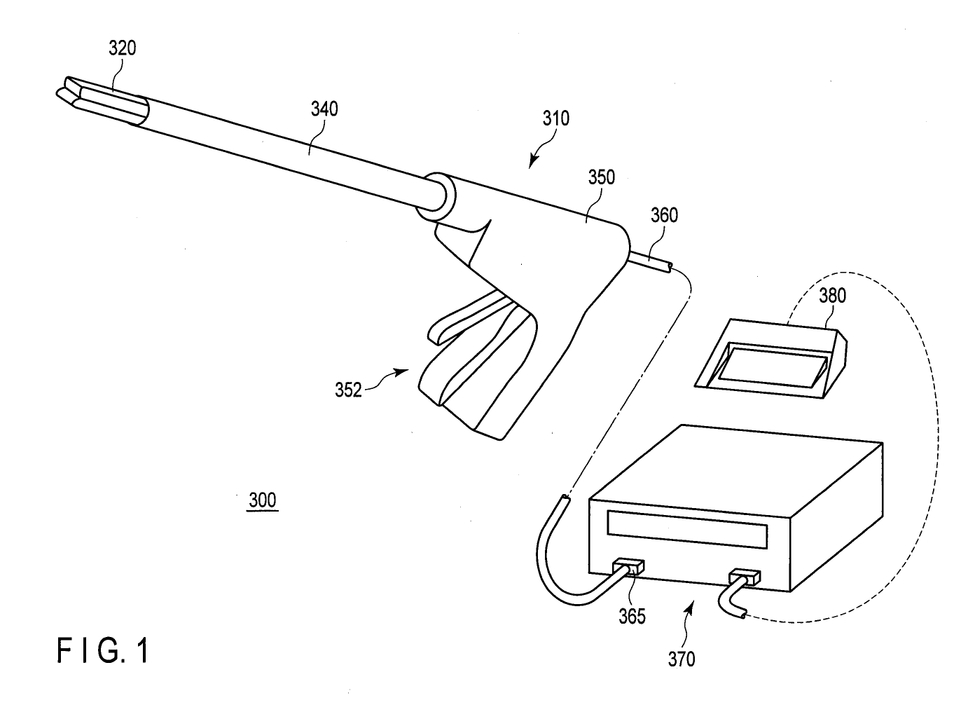

The therapeutic treatment device of the first embodiment of the present invention is used for treating a living tissue. The therapeutic treatment device applies high-frequency energy and thermal energy to the living tissue. FIG. 1 schematically shows an appearance of the therapeutic treatment device 300. As shown in FIG. 1, the therapeutic treatment device 300 includes an energy treatment tool 310, a controller 370, and a footswitch 380.

The energy treatment tool 310 is a linear-type surgical treatment tool which is penetrated through, for example, an abdominal wall, to perform treatment. The energy treatment tool 310 includes a handle 350, a shaft 340 attached to the handle 350, and a holding section 320 provided at a distal end of the shaft 340. The holding section 320 can be opened/closed, and holds the living tissue to be treated and performs treatment, such as coagulation or incision of the living tissue. In the descriptions set forth below, a portion closer to the holding section 320 will be referred to as a distal side, and a portion closer to the handle 350 will be referred to as a proximal side. The handle 350 includes a plurality of operation knobs 352 for operating the holding section 320. The handle 350 is provided with a nonvolatile memory (not shown) for storing characteristic values of the energy treatment tool 310. The shape of the energy treatment tool 310 depicted in FIG. 1 is merely an example, and the energy treatment tool 310 may of course be configured to have any other shape as long as it has a similar function. For example, the energy treatment tool 310 may be in the shape of a forceps, or the shaft may be curved.

The handle 350 is connected to the controller 370 through a cable 360. The cable 360 and the controller 370 are connected by a connector 365, and the connector is detachable. In other words, the therapeutic treatment device 300 is configured so that the energy treatment tool 310 can be replaced with another one for every treatment. The footswitch 380 is connected to the controller 370. The footswitch 380 operated by foot may be replaced with a manually operable switch or any other type of switch. The supply of energy to the energy treatment tool 310 from the controller 370 is switched on or off by an operator operating the pedal of the footswitch 380.

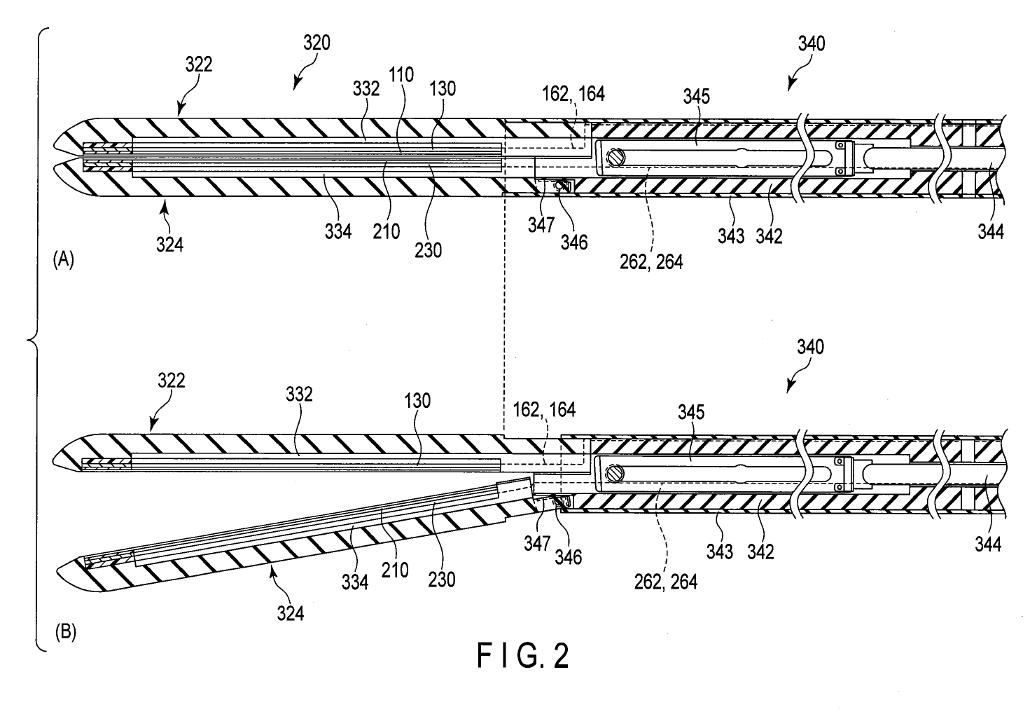

An example of the holding section 320 and shaft 340 is shown in FIG. 2. FIG. 2 (A) shows a state in which the holding section 320 is closed, and FIG. 2 (B) shows a state in which the holding section 320 is opened. The shaft 340 comprises a tubular member 342 and a sheath 343. The tubular member 342 is fixed to the handle 350 at the proximal end thereof. The sheath 343 is located on the outer side of the tubular member 342 and slidable in the axial direction of the tubular member 342.

The holding section 320 is located at the distal end of the tubular member 342. The holding section 320 includes a first holding member 322 and a second holding member 324. The proximal portion of the first holding member 322 is fixed to the distal end portion of the tubular member 342 of the shaft 340. The proximal portion of the second holding member 324 is rotatably supported by the distal end portion of the tubular member 342 of the shaft 340 by means of a support pin 346. Thus, the second holding member 324 is rotatable around the axis of the support pin 346, and opens or closes with respect to the first holding member 322.

In the closed state of the holding section 320, a cross section of the combination of the proximal portion of the first holding member 322 and the proximal portion of the second holding member 324 is, for example, circular. The second holding member 324 is urged by an elastic member 347, for example, a leaf spring, so as to open with respect to the first holding member 322. When the sheath 343 is slid to the distal side relative to the tubular member 342, and the proximal portion of the first holding member 322 and the proximal portion of the second holding member 324 are covered by the sheath 343, the first holding member 322 and the second holding member 324 are set in the closed state against the urging force of the elastic member 347, as shown in FIG. 2(A). On the other hand, when the sheath 343 is slid to the proximal side of the tubular member 342, the second holding member 324 opens with respect to the first holding member 322 by the urging force of the elastic member 347, as shown in FIG. 2(B).

A second lead line 162 connected to a first heat conduction plate 110 and a second lead line 262 connected to a second heat conduction plate 210 are inserted through the tubular member 342. A pair of first lead lines 164 connected to an electrothermal conversion element 130, which is a heat generating member (described later) arranged in the first heat conduction plate 110, is inserted through the tubular member 342. A pair of first lead lines 264 connected to an electrothermal conversion element 230 arranged in the second heat conduction plate 210 is also inserted through the tubular member 342.

A driving rod 344, connected to one of the operation knobs 352 at the proximal side, is located inside the tubular member 342. The driving rod 344 is movable in the axial direction of the tubular member 342. A cutter 345 in the form of a thin plate having an edge at the distal end is attached to the distal side of the driving rod 344. When the operation knob 352 is operated, the cutter 345 is moved via the driving rod 344 in the axial direction of the tubular member 342. When the cutter 345 is moved to the distal side, it is received in a first cutter guide groove 332 and a second cutter guide groove 334, both formed in the holding section 320.

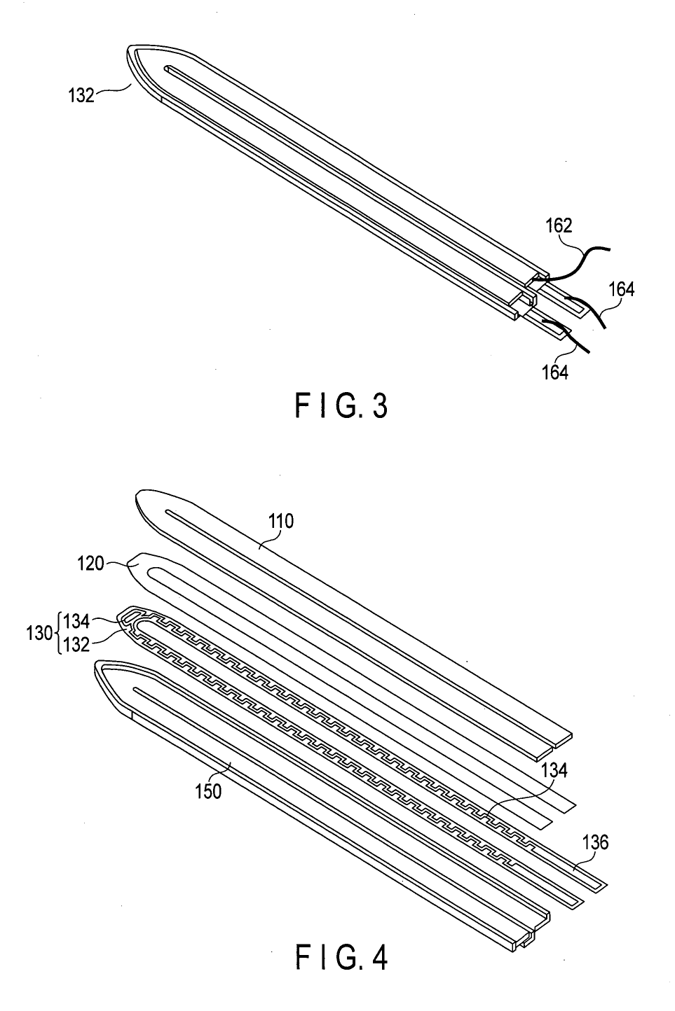

FIG. 3 shows an overall perspective view of the first holding member 322. FIG. 4 shows a configuration of the first holding member 322. The first holding member 322 has a planar shape formed into an approximate “U” shape, and a cross section formed into an approximately rectangular shape. In other words, giving primary importance to accessibility to a living tissue, the first holding member 322 has a thin plate shape, although the proximal side thereof is semicircular in accordance with the shapes of the tubular member 342 and the sheath 343. The first holding member may of course be roundish, that is, semicircular to suppress damaging a targeted living tissue when in contact with an un-targeted living tissue; however, it is preferable that the thickness of the first holding member be as small as possible. The first holding member 322 includes the heat conduction plate 110 that is brought into contact with a living tissue and transfers heat to the living tissue, an adhesive sheet 120 for adhesion, the electrothermal conversion element 130 made of an insulation substrate 132 having insulation properties and an electric resistance pattern 134 which generates heat upon receipt of electric power, a cover member 150 having insulation properties, the pair of first lead lines 164 connected to the electric resistance pattern 134 of the electrothermal conversion element 130, and the second lead line 162 connected to the heat conduction plate 110.

The electrothermal conversion element 130 is constituted by forming the electric resistance pattern 134 on the insulation substrate 132. The insulation substrate 132 is formed by a material having insulation properties. If the material having insulation properties is a resin material, such as polyimide, the insulation substrate 132 can be thin.

The electric resistance pattern 134 is formed of, for example, a stainless steel (SUS material), and a thickness thereof is, for example, 20 μm. The electric resistance pattern 134 generates heat upon receipt of electric power. The generated heat is transferred to the heat conduction plate 110 via the adhesive sheet 120. Heat generation of the electric resistance pattern 134 is controlled by the controller 370, which applies electric power for control.

A lead connecting section 136 integral with the electric resistance pattern 134 is provided on the insulation substrate 132. One end of the first lead line 164 is electrically connected to the lead connecting section 136.

The adhesive sheet 120 is a thin sheet to adhere the heat conduction plate 110 to the electric resistance pattern 134 side of the electrothermal conversion element 130. The adhesive sheet 120 has a high heat resistance, a high thermal conductivity, and electric insulation properties, and is approximately U shaped to overlie on the heat conduction plate 110, and adheres the heat conduction plate 110 and the electrothermal conversion element 130. If the adhesive sheet 120 is formed of a thermosetting member, the adhesion is achieved by heating the member.

The heat conduction plate 110 is made of a metal material having a high thermal conductivity and approximately U shaped. The heat conduction plate 110 is a member, one surface of which is heated, and another surface of which is brought into contact with a living tissue, and heats and cauterizes the living tissue. Because of the cutter guide groove, the first holding member 322 is, for example, U shaped in a plan view.

The cover member 150 is formed of a heat-resistant resin. A cutter guide groove to guide the cutter 345 is formed in the cover member 150.

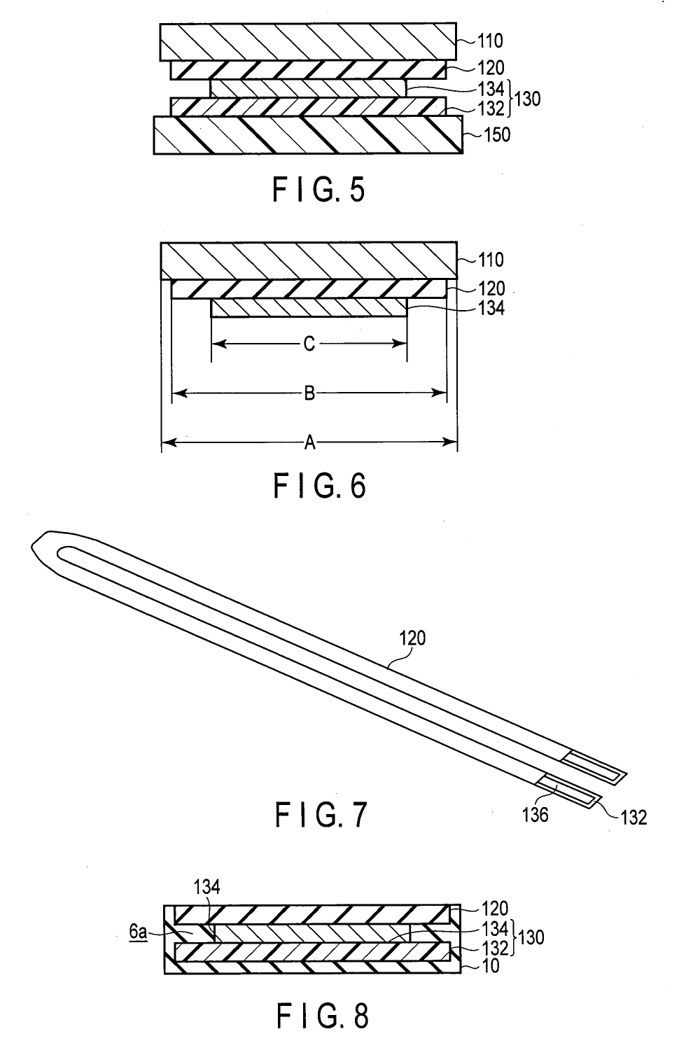

A stack state of the members forming the first holding member 322, for example, a stack state near the center in the longitudinal direction of the first holding member 322, will be explained with reference to FIG. 5, which is a cross-sectional view of the first holding member 322. First, the electric resistance pattern 134 is stacked on one surface of the insulation substrate 132. The stacked structure is called the electrothermal conversion element 130. The insulation substrate 132 may be made of any insulating material. Therefore, by using a material that can be processed to a thin substrate to form the insulation substrate 132, the first holding member 322 can be easily thinned. The adhesive sheet 120 is stacked on and adhered to the electric resistance pattern 134 side of the electrothermal conversion element 130. The heat conduction plate 110 is stacked on and adhered to a surface of the adhesive sheet 120 opposite to the electric resistance pattern 134 side. Thus, since the electrothermal conversion element 130 and the heat conduction plate 110 are adhered by the thin adhesive sheet 120, the member obtained by adhering the electrothermal conversion element 130 and the heat conduction plate 110 has a small influence on the thickness of the first holding member 322. As a result, the first holding member 322 can be easily thinned. The cover member 150 is attached to a surface of the insulation substrate 132, which is opposite to the surface of the insulation substrate 132 on which the electric resistance pattern 134 is formed. The attachment of the heat conduction plate 110 and the cover member 150 is achieved by, for example, fitting a projection (not shown) formed in the cover member 150 into a hole (not shown) formed in the heat conduction plate 110.

As described above, the first holding member 322 of the therapeutic treatment device according to the first embodiment has a configuration to enable a reduction in thickness of the first holding member 322. Specifically, the electric resistance pattern 134 on the insulation substrate 132 is attached to the heat conduction plate 110 via the adhesive sheet 120 having a high heat resistance, a high thermal conductivity, and insulation properties. Accordingly, the electric resistance pattern 134 and the heat conduction plate 110 can be arranged in close vicinity to each other. As a result, the heat generated in the electric resistance pattern 134 is efficiently transferred to the heat conduction plate 110, and reliable temperature control properties are ensured. Furthermore, the insulation substrate 132 need not be made of a material of a high thermal conductivity. Therefore, the insulation substrate 132 may be made of a resin material such as polyimide, whereby reductions in thickness and cost can be achieved. As described above, reductions in thickness and cost of the first holding member 322 can achieved.

Although the first holding member 322 has been described above, the same structure is also applicable to the second holding member 324.

Using the first and second holding members 322 and 324 that are both thin, the therapeutic treatment device 300 having the thin holding section 320 can be provided.

A therapeutic treatment device according to a second embodiment will be described with reference to FIG. 6. The second embodiment is a modification of the first embodiment. FIG. 6 shows a relationship among lengths in a transverse direction of members that constitute the first holding member 322. The dimensions of the members constituting the first holding member 322 are restricted by the relationship. The length in the transverse direction of the heat conduction plate 110 is denoted as A, the length in the transverse direction of the adhesive sheet 120 as B, and the length in the transverse direction of the electric resistance pattern 134 as C. In this embodiment, the relationship A≧B>C holds.

The length B of the adhesive sheet 120 and the length C of the electric resistance pattern 134 have the relationship B>C. Due to this relationship, the surface of the electric resistance pattern 134 is always entirely in contact with the adhesive sheet 120. Therefore, the loss of heat transferred from the adhesive sheet 120 to the electric resistance pattern 134 can be reduced.

The length A of the heat conduction plate 110 and the length B of the adhesive sheet 120 have the relationship A≧B. Due to this relationship, the surface of the adhesive sheet 120 is entirely in contact with the heat conduction plate 110. Therefore, the loss of heat transferred from the adhesive sheet 120 to the heat conduction plate 110 can be reduced. Furthermore, since the maximum area of the adhesive sheet 120 is set to a small value, the holding section 320 can be a compact configuration, which can treat a smaller part of the living tissue.

As described above, the holding member 324 of the therapeutic treatment device 300 of the second embodiment restricts the relationship among lengths in a transverse direction of the electrothermal conversion element 130, the adhesive sheet 120, and the heat conduction plate 110 used in the first embodiment. The length A of the heat conduction plate 110, the length B of the adhesive sheet 120, and the length C of the electric resistance pattern 134 in the transverse directions of the respective members have the relationship A≧B>C. Due to the relationship, the loss of heat transferred from the electrothermal conversion element 130 that generates heat to the heat conduction plate 110 that heats the living tissue can be reduced. With the above configuration, the heat efficiency of the first holding member 322, which is thinned, can be further improved.

The second holding member 324 of course has a similar structure.

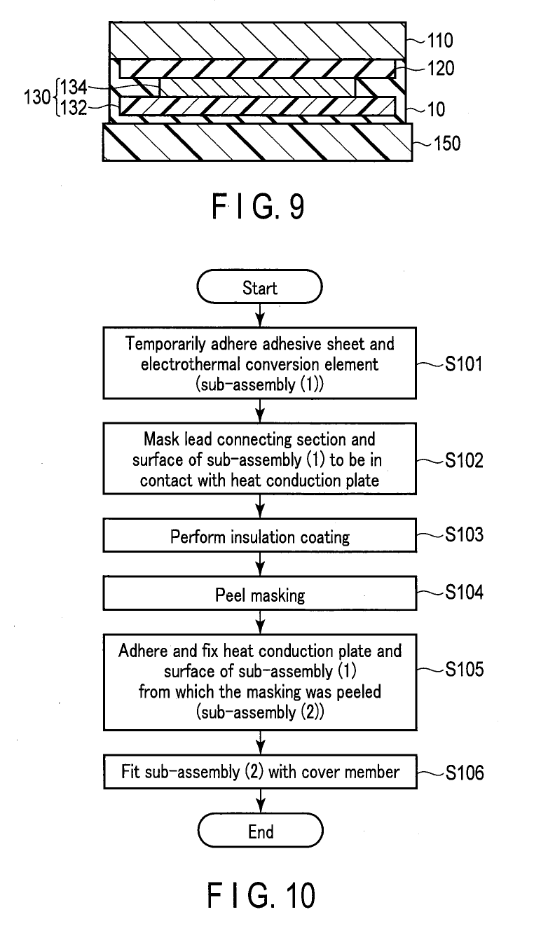

A third embodiment will be explained with reference to FIG. 7, FIG. 8, FIG. 9, and FIG. 10. The third embodiment is a modification of the first embodiment. The explanations for the same members as those of the first embodiment will be omitted. In the third embodiment, an insulator 10 is applied to cover the electrothermal conversion element 130.

Procedural steps of assembling of the third embodiment will be explained using a flowchart of FIG. 10. First, the adhesive sheet 120 and the electrothermal conversion element 130 are adhered and fixed, thereby obtaining a temporarily joined electrothermal conversion element sub-assembly as shown in FIG. 7 (step S101). The temporarily joined electrothermal conversion element sub-assembly is referred to as a sub-assembly (1). Masking is performed to mask the lead connecting section 136 and the surface of the adhesive sheet 120 opposite to the surface that is in contact with the electrothermal conversion element 130 (step S102). Thereafter, the sub-assembly (1) is entirely coated with the insulator 10 (step S103) and then the masking is peeled from the masked surface of the adhesive sheet 120 and the lead connecting section 136 to expose those surfaces (step S104). As a result of the coating, a thin film of the insulator 10 is formed around the sub-assembly (1) as shown in FIG. 8. The thin film entirely covering the sub-assembly (1) is formed at least in a gap 6a in which one surface of the electric resistance pattern 134 is exposed. The method of coating may be, for example, parylene coating, dix coating, etc. Thereafter, the exposed adhesive sheet 120 of the sub-assembly (1) is adhered to the heat conduction plate 110 (step S105). The structure obtained by adhering the sub-assembly (1) and the heat conduction plate 110 is referred to as a sub-assembly (2). Thereafter, the surface of the insulation substrate 132 side of the sub-assembly (2) is fit to the cover member 150. Accordingly, the first holding member 322 of the third embodiment configured as shown in FIG. 9 is obtained (step S106).

As described above, in the third embodiment, the sub-assembly (1), which is a part of the configuration of the first holding member in the first embodiment described above, is coated with an insulating thin film. Due to the thin film, the contact area between the heat conduction plate 110 and the thin film or the contact area between the thin film and the cover member 150 is increased. Moreover, a step due to a difference in area between members is reduced by the thin film. Accordingly, the first holding member 322, in which the adhesion between the heat conduction plate 110 and the electrothermal conversion element 130 has a high pressure resistance, can be obtained.

The second holding member 324 of course has a similar structure.

A fourth embodiment will be described. The fourth embodiment is a modification of the first embodiment. The explanations for the same members as those of the first embodiment will be omitted. In the fourth embodiment, the surface of the adhesive sheet 120 of the sub-assembly (1) that is masked in the third embodiment is also coated with an insulator 10.

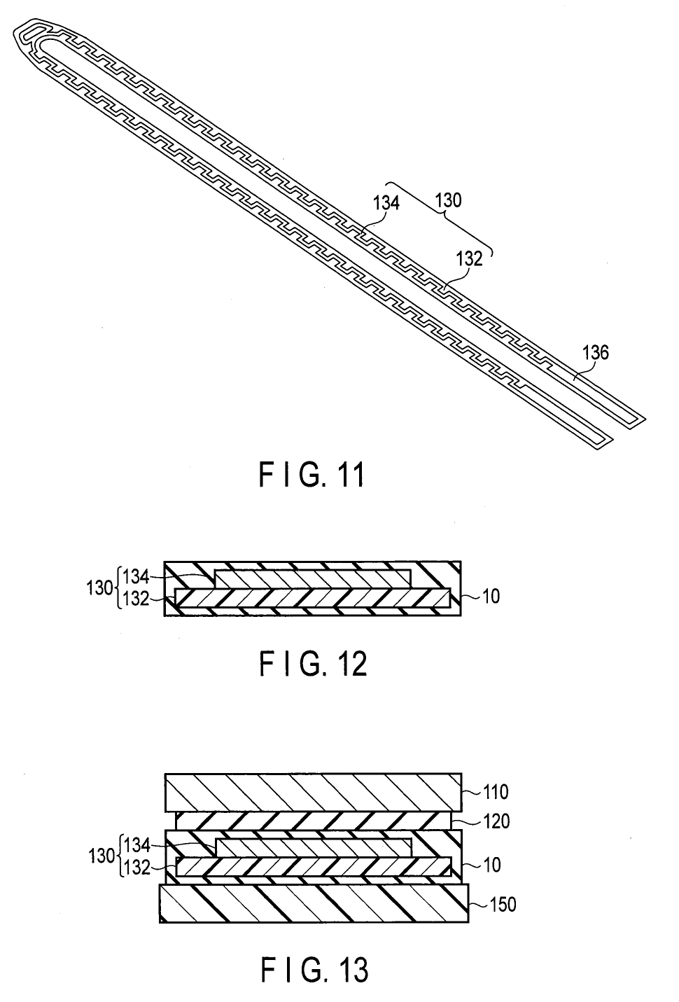

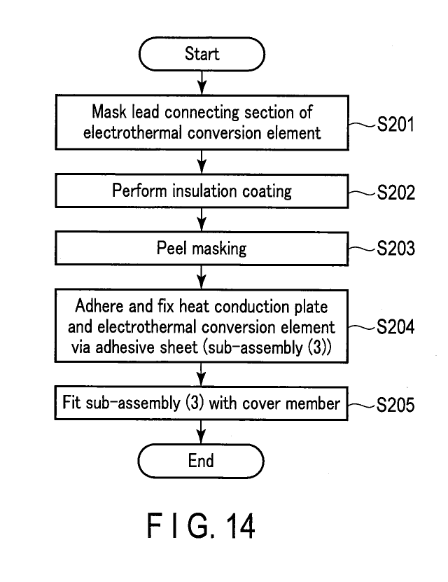

A configuration of a therapeutic treatment device 100 according to the fourth embodiment will be described with reference to FIG. 11, FIG. 12, FIG. 13, and FIG. 14. Procedural steps of assembling a first holding member 322 in the fourth embodiment will be explained using a flowchart of FIG. 14. First, a lead connecting section 136 of an electrothermal conversion element 130 as shown in FIG. 11 is masked (step S201). Thereafter, the electrothermal conversion element 130 is entirely coated with the insulator 10 (step S202). As a result of the coating, a thin film of the insulator 10 is formed around the electrothermal conversion element 130 as shown in FIG. 12. Thereafter, the masking on the lead connecting section 136 is peeled to expose the lead connecting section 136 (step S203). Then, the heat conduction plate 110 and the electrothermal conversion element 130 are adhered by the adhesive sheet 120 (step S204). The structure obtained by adhering the electrothermal conversion element 130 coated with the insulator 10 and the heat conduction plate 110 is referred to as a sub-assembly (3). The sub-assembly (3) and the cover member 150 are fit, and as a result, the first holding member 322 of the fourth embodiment configured as shown in FIG. 13 is obtained (step S205).

Thus, in the fourth embodiment of the present application, the electrothermal conversion element 130, which is a part of the components of the first holding member 322 of the first embodiment described above, is entirely coated with the insulator 10, so that a thin film of the insulator 10 entirely covers the electrothermal conversion element 130. The thin film serves to provide the first holding member 322 in which pressure resisting properties between the heat conduction plate 110 and the electric resistance pattern 134 can be higher than those in the first embodiment.

The second holding member 324 can have a similar structure.

The embodiments of the present invention have been described above. The present invention is not limited to the embodiments; various modifications or applications may be available within the scope of the gist of the invention.

For example, the shape of the first holding member 322 (and the second holding member 324) is a mere example, but is not limited to a U shape, as long as it includes a cutter guide groove. The second lead line electrically connected to the heat conduction plate may be located at a position other than that of the embodiments. For example, the second lead line may be located at the distal end of the first (second) holding member, or may be near a center in the longitudinal direction of the electrothermal conversion element 130.

The therapeutic treatment device that applies high-frequency energy and thermal energy to a living tissue was described as an example. However, the therapeutic treatment device may be a device that applies only thermal energy. In this case, the second lead line 162 is unnecessary.

Furthermore, the holding section 320 was described as a type that includes the first and second holding members 322 and 324 to hold a living tissue. However, the present invention is applicable in the same manner to a type of device that includes only one holding member and presses the heat conduction plate 110 against a living tissue for treatment.

Moreover, fitting was described as a means for connecting the sub-assembly (2) or the sub-assembly (3) to the cover member 150 as an example; however, the connecting means is not limited to this example. The sub-assembly (2) or the sub-assembly (3) may be connected to the cover member 150 by adhesive, or the heat conduction plate 110 and the cover member 150 may be joined by welding

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A therapeutic treatment device that heats a living tissue for treatment, the therapeutic treatment device comprising:
a heat conduction plate including a first principal surface that is brought into contact with the living tissue and a second principal surface opposite to the first principal surface;
an electric resistance pattern that is formed on an insulation substrate and generates heat upon receipt of electric power;
an adhesive sheet that has a high heat resistance, a high thermal conductivity, and electric insulation properties, adheres the electric resistance pattern with the second principal surface of the heat conduction plate, and transfers the heat generated from the electric resistance pattern to the heat conduction plate; and
an insulating thin film covering at least an exposed portion of the electric resistance pattern that is not in contact with the adhesive sheet and the insulation substrate.

2. The therapeutic treatment device according to claim 1, wherein:
each of the heat conduction plate, the electric resistance pattern, and the adhesive sheet has a length in a longitudinal direction and a length in a transverse direction shorter than the length in the longitudinal direction; and
the length in the transverse direction of the adhesive sheet is equal to or shorter than the length in the transverse direction of the heat conduction plate, and longer than the length in the transverse direction of the electric resistance pattern.

3. The therapeutic treatment device according to claim 1, wherein the insulation substrate is made of resin.

4. The therapeutic treatment device according to claim 2, wherein the insulation substrate is made of resin.
